(19) United States
(12) Patent Application Publication
(10) Pub. No.: US 2011/0185787 A1
(43) Pub. Date: Aug. 4, 2011

(54) INSECT HOLDER

(76) Inventors: Mathilde Briens, Harpenden (GB); David Grant, Milton Keynes (GB); Marc Bax, St Neots (GB); David Westley, Harpenden (GB); Ken Cherry, Bedford (GB)

(21) Appl. No.: 13/122,348

(22) PCT Filed: Sep. 24, 2009
(86) PCT No.: PCT/GB2009/051249

§ 371 Date: Apr. 1, 2011

(30) Foreign Application Priority Data
Oct. 1, 2008 (GB) 0817868.3

(51) Int. Cl. G01N 33/00; A01K 29/00; A01K 15/04

(52) U.S. Cl. 73/23.34; 119/6.5; 119/729

(57) ABSTRACT

An insect holder (1) for holding an insect (5) of a particular type is disclosed. The holder (1) comprises a body with a chamber (3) adapted to receive the insect (5) and a head restraint (7) for retaining the head of the insect (5) in a predetermined position. The body further comprises an electromagnetic radiation receiver (11) for monitoring intensity of an electromagnetic radiation beam so as to monitor response of the bee (5) to a target odour through the detection of an interruption of the electromagnetic radiation beam.

### TECHNICAL FIELD

This invention relates to an insect holder for holding an insect such as a bee used for the detection of at least one specific target odour.

### BACKGROUND ART

It is known that insects can have great sensitivity to odours, and can be more sensitive than known physical techniques including gas chromatography, being sensitive to odiferous materials present in very low levels down to parts per trillion in volume. Insects including bees, wasps, moths and aphids etc. can be trained or conditioned to respond with high accuracy and specificity to specific odours in a detectable manner e.g. by measuring electrical signals in antennae (electroantennography) or observing physical movement such as proboscis extensions. For example forager honey bees (species Apis mellifera) can be conditioned to a particular target odour (see e.g. Pham-Delegue H.-M. et al (1993) Behavioural discrimination of oilseed rape volatiles by the honey bee Apix mellifera L. Chem. senses 18:483-494).

In order to use insects to detect volatile substances, the insects are first loaded into individual holders, such as that described in U.S. Pat. No. 7,036,454. These holders allow the insect to be trained either by hand or automatically to an odour of interest. In the case of bees, once trained, the bee will extend its proboscis (Proboscis Extension Reflex—PER) upon detection of a trained odour signalling that the trained substance is present. This PER response must be monitored both during training of the insect and whilst it is being used for the detection of the volatile substance. Currently insects are trained by hand and their responses are only monitored by the human eye or cameras. U.S. Pat. No. 7,237,504 describes one method of monitoring the response of the insect to an odour by an image analysis technique, using a CCD camera to record the movement.

One disadvantage of the system described in U.S. Pat. No. 7,237,504 is that cameras are very difficult to miniaturize in order to achieve a portable detection device using for instance, more than three bees. Furthermore, cameras are expensive.

It is an object of this invention to provide an improved system which solves or alleviates the problems associated with the prior art.

### DISCLOSURE OF THE INVENTION

According to a first aspect of the invention, there is provided an insect holder for holding an insect of a particular type, the holder comprising a body with a chamber adapted to receive the insect and a head restraint for retaining the head of the insect in a predetermined position, wherein the body further comprises an electromagnetic radiation transmitter or receiver.

Preferably the body of the holder comprises the receiver which is an infrared receiver.

Preferably, the electromagnetic receiver or transmitter is arranged within the body in such a way that, in use, a path of an electromagnetic beam to the receiver from the transmitter is interrupted by motion of part of the insect. When the holder is in use with a bee the receiver is arranged such that, in use, the beam is interrupted by motion of the proboscis and/or the antennae of the bee.

Preferably, the body of the holder has a first part in which the chamber is located and a second part having a recess in which is accommodated the receiver.

Preferably the second part of the body has a front wall which is adjacent in use to the head of the insect, the front wall is provided with an aperture and the receiver is positioned at one end of the aperture.

The receiver may be mounted to a printed circuit board which is mounted within the body of the holder so as to form the bottom of the recess. The recess may further be made water tight by a seal arranged at the opposite end of the aperture.

Preferably the first part is an upper part of the body and the second part is a lower part of the body.

In a variant, the body may further comprise a heating element to maintain the insect above a predetermined temperature.

Preferably, the heating element is located within the recess.

In another variant, the body may comprise a data storage device.

Preferably, the data storage device is a random-access memory which is located within the recess.

Preferably, the holder further comprises communicating means to communicate with a remote unit.

In a further variant, the body comprises a receiver and a transmitter. Preferably the transmitter is an infrared LED.

According to a second aspect of the invention, there is provided an odour detecting device for use with at least one insect holder in accordance with the first aspect of the invention, when only provided with one of a transmitter or a receiver, comprising for each insect holder a corresponding transmitter or receiver so as to form with the receiver or transmitter of each holder a monitoring system for detecting a response of the insect in the holder to a target odour.

According to a third aspect of the invention, there is provided an odour detecting device for use with at least one insect holder in accordance with the first aspect of the invention, when provided with an integrated transmitter, comprising communication means to connect each holder to a remote unit.

### BRIEF DESCRIPTION OF THE DRAWINGS

The invention will now be described by way of example with reference to the accompanying drawings of which;

FIG. 1 is a schematic side view of an insect holder according to the invention;

FIG. 2 is a cut away front view of the holder of FIG. 1, seen from the right hand side; and

FIG. 3 is a schematic side view of the insect holder of FIG. 1 in use with a transmitter, forming hence an odour detecting device.

### MODE(S) FOR CARRYING OUT THE INVENTION

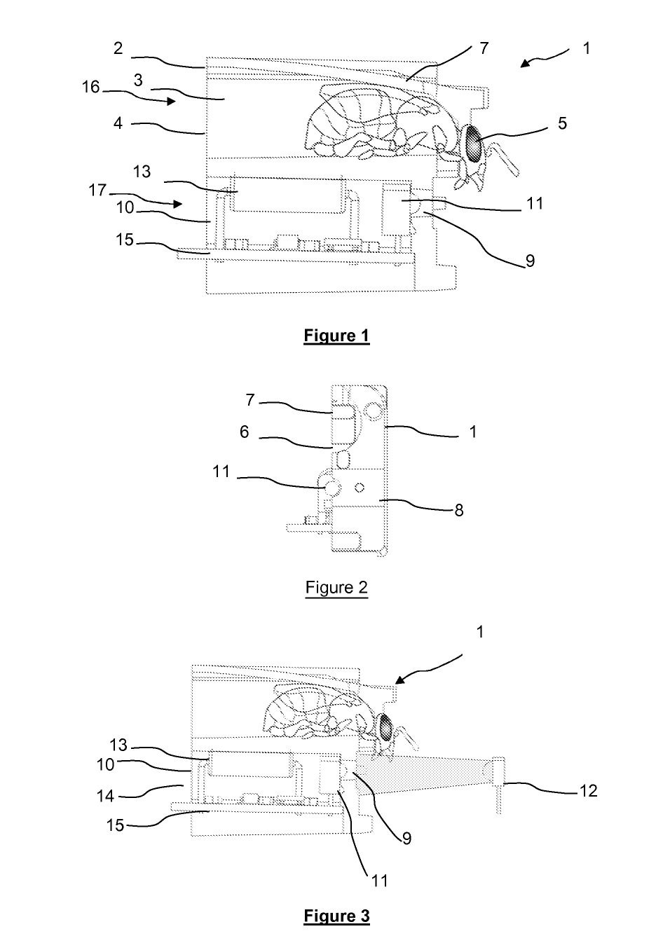

With reference to the FIGS. 1 to 3, there is shown an insect holder 1 made from a one piece plastic moulding. The insect holder has a box like form body. The body of the holder comprises two parts; an upper part 16 and a lower part 17. The upper part comprises a housing section 2 having a chamber 3. The housing has an opening at one end defining an inlet 4 to the chamber through which an insect 5, such as a forager honey bee, is to be inserted into the chamber (as will be further explained in details) and an opening at the other end defining a head opening 6 for the bee. The housing 2 comprises a head restraint 7 to trap the bee in a predetermined position, for instance as known from U.S. Pat. No. 7,036,454 or as disclosed in Batson et al, Journal of Comparative Psychology, 1992, Vol 106, No 2, 114-119; Shafir et al, Animal Behaviour, 1999, 57, 1055-1061; Buckbee et al, Journal of Insect Behaviour, Vol 10, No 4, 1997, 479-491 and Abramson et al, J Entomol. Sci. Vol 36, No 1 (2001), in which the head of the bee is visible so that observations can be made on the bee head (e.g. monitoring proboscis extension in known manner in response to a target odour).

The head restraint 7 comprises a flexible tongue having a barb at its free end arranged to engage with the back of the insect's head so that the head of the insect protrudes through the head opening 6 as shown in FIG. 1 and prevents the bee from leaving the chamber 3 via the inlet.

The lower body part of the holder comprises a recess defined by a front wall, and two opposing side walls. The front wall 8 which is provided with an aperture 9 opening into a recess 10 of the lower part of the body. At one end of the aperture 9 is mounted a receiver 11, such as an electromagnetic radiation receiver, preferably an infrared phototransistor, in such a way that the receiver 11 closes the aperture 9.

As shown in FIG. 3, the electromagnetic radiation receiver 8 is associated, in use with an electromagnetic radiation transmitter 12 such as an infrared light emitting diode (LED). The receiver and transmitter form a monitoring system. The transmitter 12 is located outside the holder and produces a constant beam across the aperture 9 onto the receiver 11.

The aperture 9 and the receiver 11 are located within the body in such a way that any extension of the proboscis of the bee interrupts the beam produced by the transmitter 12. In other words the aperture 9 is arranged such as any extension of the proboscis of the bee reaches at least the major part of the aperture 9.

In use, the transmitter 12 produces a constant beam onto the receiver, when the bee responds to the presence of a target odour, the bee extends its proboscis which causes the beam transmitted to be at least partially interrupted. Accordingly the presence/absence of a Proboscis Extension Reflex (PER) of the bee in the beam may be monitored based on the intensity of the beam detected by the receiver 11. The electromagnetic radiation transmitted and detected by the receiver can be, but is not limited to, visible, infrared and/or UV light.

As well as being used to detect the presence of a target odour the holder is also used for monitoring the vitality of the insect, by detecting movement of part of the insect. For example movement of a bee's antennae can lead to changes in the reading by the receiver 11. The changes in reading by the receiver 11 caused by movements of the antennae are generally of a lower amplitude and/or shorter time scale than that caused by the PER. Therefore the two distinct effects can be distinguished from each other. The detection of antennae movement shows that the bee is still alive. If prolonged absence of antennae movement is detected a warning signal is sent to the end user that the bee in that particular holder is dead.

Located in the recess 10, the holder further includes a heating element 13. The heating element 13 comprises at least one resistive element. In this example two resistive elements which are located adjacent to the bottom surface of the chamber. The heating element 13 also comprises means for providing a voltage in the resistive element and means for increasing the voltage so as to increase or decrease the temperature of the resistive elements. The heating element 13 raises the temperature of the surfaces which the insect is in contact with in the chamber so that the bee can be kept at its working temperature, i.e. above approximately 15° C.

The heating element 13 formed as part of (or integral to) the holder reduces the risk of contamination that may occur, for example, when blowing warm air around the housing environment such as described for example in U.S. Pat. No. 7,237,504.

The heating element 13 is preferably associated with a temperature sensor (not shown). The temperature sensor provides an indication of the temperature of the bottom surface of the chamber. An electronic control circuit integrated in the recess regulates the heat dissipated from the heating element so as to achieve a constant desired temperature.

Each temperature sensor of each insect holder detects the environmental conditions of each bee. If the temperature is considered to fall below a minimum temperature required for effective use of the bee, the voltage system in the resistive element is increased in the corresponding insect holder to heat up the contact surface of the holder, i.e. the bottom surface of the upper part, and maintain the bee at the required temperature.

In a variant of the present invention, the bee holder also comprises a storage device to store data such as a unique identification code which is relevant to the bee to be held. The data is stored in electronic form and data can be written, stored and read to the device. The storage device is preferably associated with a microcontroller and has a random-access-memory (RAM) which stores data to uniquely identify each insect holder and its history. For example, each individual holder is assigned a unique identification code before its first use. Using the identification code the history of each loaded bee can be traced from loading through conditioning, deployment and post-deployment unloading.

Additional data regarding the bee can also be stored within the storage device of each individual bee holders.

Examples of information that can be stored include date and time of capture; date and time of loading, colony identifier, date and time of conditioning regime, substances conditioned for, performance of the bee during conditioning, operator of the detecting device, location of use and/or any other useful information.

The information stored in the bee holder can be encrypted. This is useful to prevent tampering with the information.

The insect holder includes connection means to allow the holder to communicate with a remote unit, such as a computer, which can include a central database comprising further information about the bee. Examples of connection means which allow communication to and from the insect holder include direct galvanic contact, optical communication means, radio communication means etc.

It will be noted that the recess 10 is made water tight via a seal 14 at the opposite end of the aperture 9 to protect the above described components i.e. heating element, receiver, and/or storage device which are all connected to a printed circuit board 15. The printed circuit forms the bottom surface of the recess 10. This allows the holder to be washed between detection cycles to minimise contamination of the holder from earlier detection cycles.

In use, the bee is inserted in the chamber through the opening in the housing. The bee is inserted manually or automatically. In automatic insertion the bee enters the housing via a loading tube that is inserted into the holder and maintains the head restraint 7 in an open position such that the head of the insect can protrude through the second opening 6 in the chamber 3. When the bee has fully entered the chamber and is in the correct position, the tube can be removed releasing the head restraint 7 to close over the insect and to trap the insect in the chamber so that the insects head protrudes from the chamber.

A plurality of insect holders can be secured together in a holding unit (not shown) or directly to the detection unit via connecting means. The holding unit is then used with a detector unit. The detection unit comprises associated transmitters and an associated air flow system and gas sample supply system. The holding unit is inserted into the detection unit. A gas sample to be tested is supplied to the insects via a sample injection system.

The insect's response to the gas is monitored by the electromagnetic radiation sensors 11 in each insect holder. If the bee detects the target odour in the sample, a PER response is detected by the sensors due to the PER causing an interruption of the beam reaching each sensors from each transmitter 12.

The electronic components; i.e. heating device, transmitter, receiver and/or storage device are powered by an internal power supply, such as an internal battery located in the body of the insect holder. Alternatively the components can be powered by an external power source, the insect holder having connection means to connect the electronic components of the insect holder to the external power source. The detection unit can comprise the power supply.

The monitoring system described herein has the advantage of being able to be miniaturised for integration within the individual bee holder itself and to allow ease of alignment with the proboscis.

Although the insect holder has been described by way of example with reference to a specific embodiment of the invention, various modifications or additions may be made without departing from the invention. For example, it will be appreciated that the electromagnetic radiation transmitter could be attached to the insect holder and formed part of the body of the holder. In a modification the transmitter is located within the insect holder and the receiver is located outside the holder, to form the monitoring system. In another modification the two parts of the body may be arranged such that an upper part of the body of the insect holder comprises the heating element and/or storage device and a lower part of the body may comprise the chamber. In a further modification the storage device is a passive storage device, such as bar code. The bar code containing information relevant to the insect, such as a unique identification code specific to the insect in the holder.

1. An insect holder for holding an insect of a particular type, the holder comprising;
a body with a chamber adapted to receive the insect;
a head restraint for retaining a head of the insect in a predetermined position; and
an electromagnetic radiation transmitter or receiver.

2. An insect holder according to claim 1 wherein the electromagnetic radiation transmitter or receiver comprises an infrared receiver.

3. An insect holder according to claim 1, wherein the electromagnetic radiation receiver or transmitter is arranged within the body in such a way that, in use, a path of an electromagnetic radiation beam to the receiver is interrupted by motion of part of the insect.

4. An insect holder according to claim 3 when in use with a bee the receiver is arranged such that, in use, the beam is interrupted by motion of a proboscis and/or an antennae of the bee.

5. An insect holder according to claim 1 wherein the body of the holder has a first part in which the chamber is located and a second part having a recess in which is accommodated the electromagnetic radiation transmitter or receiver.

6. An insect holder according to claim 5, wherein the second part of the body has a front wall which is adjacent in use to the head of the insect, the front wall is provided with an aperture and the electromagnetic radiation transmitter or receiver is positioned at one end of the aperture.

7. An insect holder according to claim 6, wherein the electromagnetic radiation transmitter or receiver is mounted to a printed circuit board which is mounted within the body of the holder so as to form a bottom of the recess, the recess is further made water tight by a seal arranged at the opposite end of the aperture.

8. An insect holder according to claim 5 wherein the first part is an upper part of the body and the second part is a lower part of the body.

9. An insect holder according to claim 5 wherein the body further comprises a heating element to maintain the insect above a predetermined temperature.

10. An insect holder according to claim 9, wherein the heating element is integrated to the body of the holder.

11. An insect holder according to claim 9 or in which the heating element comprises at least one resistive element, voltage means and voltage variance means.

12. An insect holder according to claim 9 further comprising a temperature sensor.

13. An insect holder according to claim 9 wherein the heating element is located within the recess.

14. An insect holder according to claim 1 wherein the body comprises a data storage device.

15. An insect holder according to claim 14 wherein the storage device is random-access memory.

16. An insect holder according to claim 14 wherein the storage device is located within the recess.

17. An insect holder according to claim 1 further comprising communication means to communicate with a remote unit.

18. An insect holder according to claim 1 in which the body comprises a receiver and a transmitter.

19. An insect holder according to claim 18, wherein the transmitter is an infrared LED.

20-22. (canceled)

23. An assembly comprising:
at least one insect holder for holding an insect of a particular type, the insect holder including:
a body with a chamber adapted to receive the insect;
a head restraint for retaining a head of the insect in a predetermined position; and
an electromagnetic radiation transmitter or receiver; and
an odor detecting device for use with the at least one insect holder, the odor detecting device including a corresponding transmitter or receiver so as to form with the receiver or transmitter of the at least one holder a monitoring system for detecting a response of the insect in the holder to a target odor.

24. The assembly of claim 23, further comprising communication means to connect the one or more insect holders to a remote unit.

25. An assembly comprising:
at least one insect holder for holding an insect of a particular type, the insect holder including:
a body with a chamber adapted to receive the insect;
a head restraint for retaining a head of the insect in a predetermined position; and
an electromagnetic radiation transmitter and receiver disposed within the body; and
an odor detecting device for use with the at least one insect holder, the odor detecting device including connecting means for attaching the at least one insect holder to a remote unit.
